(19) United States
(12) Patent Application Publication
(10) Pub. No.: US 2014/0251340 A1
(43) Pub. Date: Sep. 11, 2014

(54) PATIENT INTERFACE DEVICE FOR BEING CONNECTED TO A PATIENT'S RESPIRATORY SYSTEM AND ASSOCIATED METHODS

(71) Applicant: KONINKLIJKE PHILIPS N.V., EINDHOVEN (NL)

(72) Inventors: Sander Theodoor Pastoor, Utrecht (NL); Krijn Frederik Bustraan, Eindhoven (NL); Timon Rutger Grob, Geldrop (NL); Rudolf Maria Jozef Voncken, Eindhoven (NL)

(21) Appl. No.: 14/352,764

(22) PCT Filed: Oct. 17, 2012
(86) PCT No.: PCT/IB2012/055659

§ 371 Date: Apr. 18, 2014

Related U.S. Application Data
(60) Provisional application No. 61/549,884, filed on Oct. 21, 2011.

(51) Int. Cl. A61M 16/06

(52) U.S. Cl. CPC A61M 16/0683; A61M 16/0666; A61M 16/0622
USPC 128/207.18

(57) ABSTRACT

A patient interface device is described for delivering a gas to a patient, the patient interface comprising a fastening means for fastening the patient interface to the user, the fastening means consisting of an element adapted for engaging the user's nose during use, wherein the fastening means is adapted for being fixed to said nose by exerting a pressure on said nose substantially in the width direction of said nose; and associated method.

### FIELD OF THE INVENTION

The present invention relates to patient interface devices for being connected to a patient's respiratory system in order to provide a flow of gas to an airway of the patient, and associated methods.

### BACKGROUND OF THE INVENTION

There are numerous situations where it is necessary or desirable to deliver a flow of breathing gas non-invasively to the airway of a patient, i.e., without intubating the patient or surgically inserting a tracheal tube in the esophagus. For example, it is known to ventilate a patient using a technique known as non-invasive ventilation. It is also known to deliver continuous positive airway pressure (CPAP) or variable airway pressure, such as a bi-level pressure that varies with the patient's respiratory cycle or an auto-titrating pressure that varies with the monitored condition of the patient, to the airway of a patient/user. Typical pressure support therapies are provided to treat a medical disorder, such as sleep apnea syndrome, in particular, obstructive sleep apnea (OSA) or congestive heart failure and/or other medical and respiratory disorders, such as Cheynes-Stokes respiration, congestive heart failure, and stroke.

Non-invasive ventilation and pressure support therapies involve the placement of a patient interface patient interface device, which is typically a nasal or nasal/oral mask, on the face of a patient to interface the ventilator or pressure support system with the airway of the patient so that a flow of breathing gas can be delivered from the pressure/flow generating patient interface device to the airway of the patient. It is known to maintain such masks on the face of a patient by a headgear having upper and lower straps, each having opposite ends threaded through connecting elements provided on the opposite sides and top of a mask.

Because such masks are typically worn for an extended period of time, a variety of concerns must be taken into consideration. For example, in providing a pressure support therapy to treat OSA, the patient normally wears the patient interface patient interface device all night long while he or she sleeps. Patient interface development has generally involved balancing of two competing goals: a) secure attachment to and seal with the user's face to create an airtight seal in order to facilitate the required positive airway pressure, and b) comfort to the user in order to maximize patient compliance, i.e., usage of the medical therapy. An airtight seal can be achieved by tightening the mask down firmly against the patient's face. However, this solution oftentimes results in discomfort to the user due to relatively high strapping forces needed to ensure a secure seal against the patient and less than satisfactory patient compliance. Alternatively, the mask may be fit loosely on the patient's face to enhance comfort. However, the effectiveness of the mask may be compromised if it is too loose.

In EP 0658356 a nasal positive airway pressure patient interface device is provided having a pair of nasal members each having a cannula tip to be inserted into the nares of the patient. The nasal members are kept in to position by means of e.g. a head band, a foam pad and an adjustable support strap extending from the head band to aid in holding the nasal members adjacent the nose of the user. This solution provides the patient with little comfort, and is very tedious and labour-intensive in use.

### SUMMARY OF THE INVENTION

It is an object of the present invention to provide a patient interface device for being connected to a patient's respiratory system in order to provide a flow of gas to an airway of the patient, which overcomes the shortcomings of the prior art.

The above objective is accomplished by a method and patient interface device according to the present invention.

Particular and preferred aspects of the invention are set out in the accompanying independent and dependent claims. Features from the dependent claims may be combined with features of the independent claims and with features of other dependent claims as appropriate and not merely as explicitly set out in the claims.

According to a first aspect of the present invention, a patient interface device for delivering a gas to a patient is disclosed, the patient interface comprising a fastening means or device for fastening the patient interface to the user, the fastening means or device consisting of an element adapted for engaging the user's nose during use.

The patient interface device can be any device for delivering a gas to a patient. It can for instance comprise a respiratory mask. It can comprise an adaptor means or device as explained further below. Typically the patient interface comprises one or more gas supply means or devices as for instance tubes which are adapted for transporting the respective gas. The gas can for instance be air, or could be air mixed with for instance a medicine or anesthetic. The gas can for instance be provided at room temperature, but also other temperatures which can suitable for the patient are possible. The one or more tubes are preferably rigid such that they do not substantially deform when a pressure which is higher or lower than the ambient pressure (for instance atmospheric pressure) is provided inside said tubes. The tubes can for instance be suitable for performing CPAP treatment.

It is an advantage of embodiments of the present invention that the fastening means does less or not disturb the patient as many state of the art patient interfaces would do. It is a further advantage that the patient can wear the interface when sleeping.

It is a further advantage that not necessarily obtrusive head straps are required for fastening the patient interface device to the patient.

According to advantageous embodiments, the fastening means is adapted for engaging only with the nose of the patient.

It can for instance engage with the outer surface of the sidewalls of the nose, with the internal surface of the nose (for instance inner surface of the sidewalls of the nose, or for instance with the inner surface defined by the “separator” between the two nasal passages of the nose, the nasal septum), with the columella, or with other nose members; or with any combination of these.

It can for instance only engage with the outer surface of the sidewalls of the nose, only with the internal surface of the nose (for instance inner surface of the sidewalls of the nose, or for instance with the inner surface defined by the “separator” between the two nasal passages of the nose, the nasal septum), only with the columella, or only with any combination of these.

According to preferred embodiments, the fastening means is adapted for being fixed to the nose by exerting a pressure on the nose substantially in the width direction of the nose. The pressure can be exerted on the outer surface of the sidewall of the nose, but can also be exerted on the inside of the nose (for instance on inner surface of the sidewalls of the nose, or for instance on the inner surface defined by the nasal septum), or on both of inner (any of the above mentioned examples) and outer surfaces of the sidewalls of the nose.

According to preferred embodiments, the fastening means comprises a nose clamp.

According to preferred embodiments, the fastening means comprises nose pads for being brought in contact with the lateral external sidewalls of the nose.

According to preferred embodiments, the nose pads are inflatable, such that when they are inflated the exerted pressure on the nose is substantially larger than when they are not inflated. These embodiments bring the advantage that after a first preliminary fastening, an improved fastening can be achieved by inflating the nose pads. The inflatable nose pads may be adapted to have also a larger width and/or thickness when inflated than when not inflated. The gap defined between the nose pads (which can also be defined by other patient interface device elements, as for instance an arch which will be described further on) is then larger when the nose pads are not inflated, such that an accommodation of the patient interface device on the nose of the patient can more easily be achieved. It is an advantage that inflatable nose pads also provide an optimized distribution of the exerted pressure on the nose, contrary to rigid nose pads. This may also result in more comfort.

According to preferred embodiments, contact surfaces of the nose pads, for being brought in contact with the outer nose skin, are adapted for providing improved friction. This may increase the threshold for loosing grip on the nose, and result in an improved fastening. Alternatively or additionally, the contact surfaces of the nose pads, for being brought in contact with the outer nose skin, may comprise an adhesive. The presence of the additive is though not necessary.

According to preferred embodiments, the patient interface device further comprises an adaptor means or device (e.g. an adaptor) for providing a coupling between a gas supply means and the nasal passages of the respiratory system, and the fastening means is adapted for fixing the position of the adaptor means. Although not strictly necessary, the coupling between the gas supply means and the nasal passages of the respiratory system can be substantially airtight. This is certainly the case when pressure has to be controlled of the airflow provided to the patient (CPAP applications), or when medicine or anesthetic doses needs to be controlled.

According to preferred embodiments, the patient interface device can further comprise an arch part which is on its outer ends connected to the nose pads, and wherein the arch part further comprises a support portion adapted for supporting the adaptor means.

The arch part can be a typical connector of the nose pads. It can at the same time also function as a support for the adaptor means.

According to preferred embodiments, the arch part is rotatably mounted along an axis defined by a straight line connecting the nose pads, and is shaped such that it can freely rotate over a tip of the nose, and such that it can be brought in a fixing position below the nose where it can support the adaptor means.

According to preferred embodiment, the arch part is substantially rigid, and each of the two nose pads is rotatably mounted to the arch part, such that it can rotate or at least make a pivotal movement along respective rotation axis. According to preferred embodiment the rotation axis of a first and a second nose pad are substantially the same or the same.

According to preferred embodiments, the arch part is fixed to and rotatably mounted to the adaptor means, preferably near its support portion.

According to preferred embodiments, the arch part is mainly rigid or semi rigid, except for its outer ends which are abutting on the nose pads or forming the nose pads, the outer ends being flexible and elastic. The flexible and elastic outer ends may contribute in exerting a pressure on the nose and allow an easy accommodation of the patient interface device on the nose.

According to preferred embodiments, the adaptor means comprises a pair of cannula to be inserted into the nasal passages of the patient, the cannula being at least partially rigid, the nose clamp (fastening means) and the cannula being adapted for cooperating in positioning and fixing the adaptor means.

It is an advantage of these embodiments, that the cannula can provide an efficient coupling with the nasal passages of the patient, as they can be inserted in these nasal passages. The fact that the cannula are preferably partially rigid, provides the advantage that a pressure exerted on the outer surface of the sidewalls of the nose, for instance in substantially the width direction of the nose, can be built up without substantially deforming the nasal passages of the patient, as this pressure is countered by the rigid (part of) the cannula. The sidewalls of the nose can thereby be squeezed between the nose pads and the cannula. This can cause a fastening of the patient interface device.

According to preferred embodiments, the cannula are connected by an interconnect portion. The interconnect portion preferably comprises an upper surface which is adapted for resting against the columella of the nose.

These embodiments provide the further advantage of easier manipulation of the set of cannula (a common manipulation), as well as an automatic regulation as to how far the cannula are allowed or should enter the nasal passages. The interconnect portion can preferably be rigid, but could also be flexible. The advantage of a rigid interconnect portion is that it can define a predetermined distance between the cannula.

The interconnect portion, and also the cannula can thus be adapted for use with a specific patient, for instance by taking into account the dimensions of the nose and nasal passages of the patient.

According to preferred embodiments, the adaptor means is detachable from the fastening means. This allows the use of a (more) generic fastening means with a specific adaptor means for a patient and the reuse of the fastening means (e.g. nose clamp). It also allows the insertion of the adaptor means before fastening it with the fastening means.

According to preferred embodiments, the cannula are at least partially inflatable. The cannula can for instance comprise an inflatable cuff. This has the advantage that the cannula can be inserted more easily into the nasal passages of the patient, before being inflated. After inflation, the cuffs can cause the coupling between the cannula (and thus adaptor means) and the nasal passages of the client to become airtight, and moreover the fastening can be improved. The partially inflatable part may also provide improved comfort for the patient, as compared to a rigid version.

According to preferred embodiments, the inflatable cuff comprises cuff apertures in the cannula such that the cuffs can be inflated by a positive airway pressure generated during inhalation and exhalation. This embodiment provides the advantage that the pressure, or pressure provisioning means, which is already provided for other purposes (for instance CPAP applications) can also be reused for inflating the cuffs.

According to preferred embodiments, the inflatable cuff can be inflated independently from any generated positive airway pressure.

According to preferred embodiments, the patient interface device further comprises a removable cap covering the fastening means. These embodiments provide the advantage that comfort and manipulability of the device is improved. The presence of such a cap surface can improve the stability of the interfacing device, as it becomes more difficult penetrate or pull at any of its component with objects external to the device. It also reduces the possibility that the patient would unconsciously remove (part) of the mask or hoses when sleeping.

According to preferred embodiments, the fastening means comprises a structure for being attached within the nose. It may be adapted for attachment to the nose by means of a hook or other inner nose attachments means or device. Alternatively it may be adapted for attachment to the nasal septum. It may be adapted for attachment further in the nose, behind the nasal septum.

According to a second aspect of the present invention, a method for delivering a gas to a patient by means of a patient interface is disclosed, comprising fastening the patient interface by engaging the user's nose.

According to preferred embodiments, the method comprises fastening the patient interface by exerting a pressure on the nose substantially in the width direction of the nose.

According to preferred embodiments, the method comprises further comprises positioning an adaptor means for providing a coupling between a gas supply means and the nasal passages of said respiratory system, and fastening the position of said adaptor means.

In the above, features and advantages corresponding to embodiments of the first aspect of the present invention are supposed to be also disclosed, mutatis mutandis, for the second aspect of the present invention, and vice versa.

The teachings of the present invention permit the design of improved patient interface device for being connected to a patient's (10) respiratory system in order to provide a flow of gas to an airway of the patient, which is much more comfortable to the patient and easy in use. The fact that only the nose serves as a sole anchor for the patient interface device solves many of the problems of state of the art systems, which are much more complex because they involve attachment or guidance along other parts of the head of the patient.

### BRIEF DESCRIPTION OF THE DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 is a schematic representation of a perspective view of an embodiment of the present invention.

FIG. 2 is an enlarged fragmentary view showing more detail of the patient interface device of FIG. 1.

FIG. 3 is a schematic representation of a perspective view of an adaptor means, or part of an adaptor means, according to embodiments of the present invention.

FIG. 4 is a schematic representation of a perspective view of another adaptor means, or part of an adaptor means, according to other embodiments of the present invention.

FIG. 5 is a schematic representation of a perspective view of another embodiment of the present invention.

FIG. 6 is a schematic representation of a front planar view of a patient interface device according to embodiments of the present invention, comprising a force diagram.

FIG. 7 is a schematic representation of a perspective view of another embodiment of the present invention.

FIG. 8 is a schematic representation of a perspective view of another embodiment of the present invention.

In the different figures, the same reference signs refer to the same or analogous elements.

### DETAILED DESCRIPTION OF THE EMBODIMENTS

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes.

Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

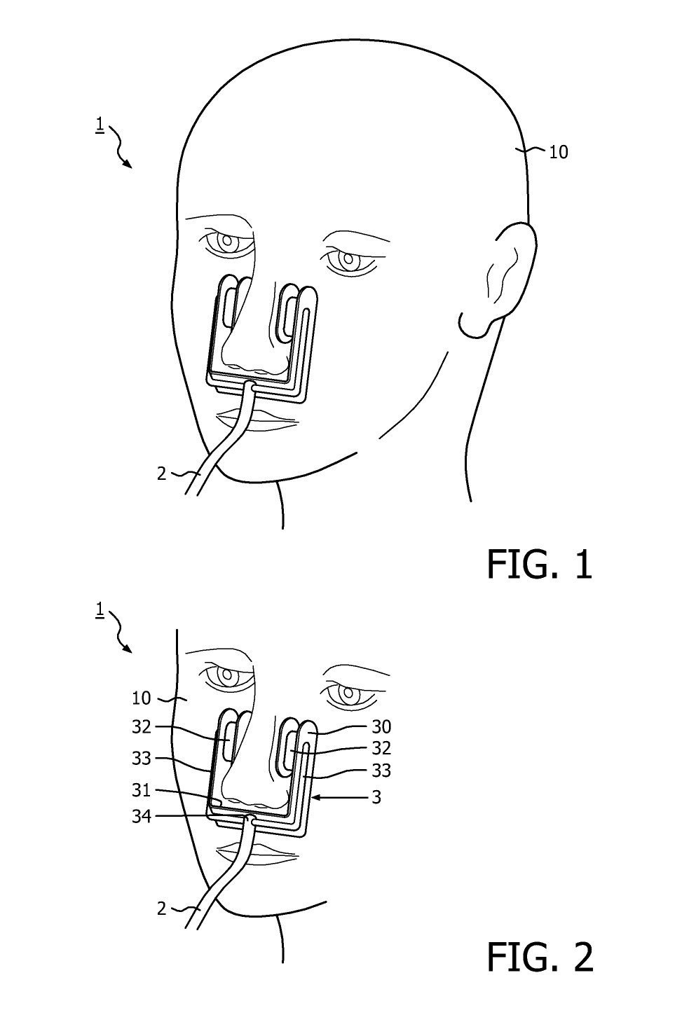

In FIG. 1 a patient interface device 1 is depicted for being connected to a patient's 10 respiratory system in order to provide a flow of gas to an airway of the patient. The patient interface device 1 comprises an adaptor means 4 (not visible), as for instance a rigid nostril insert 41, for providing an substantially airtight coupling between a gas supply means 2, for instance a hose guiding the gas towards the patient, and the nasal passages of the respiratory system, and a fastening means 3 adapted for fixing the position of the adaptor means 2, wherein the fastening means 3 is further adapted to be attached only to the nose of the patient 10. In this example embodiment the fastening means is embodied as a nose clamp. The nose clamp is adapted for being fixed to the nose on one side on to support the adaptor means on the other side.

FIG. 2 is an enlarged fragmentary view of FIG. 1, showing more detail of the nose clamp 3. A hose 2 is guiding gas towards the nose of the patient, into which the gas is led by means of the adaptor means (not depicted). The nose clamp 3 comprises nose pads 32 which are adapted for resting against the outer sidewalls of the nose, and which can be seen as “anchors” of the fastening means 3, and thus of the patient interface device 1. The nose pads 32, which are connected to the outer ends of an arch part 30, and which are thus connected with each other, fix the fastening means to the nose by exerting a pressure or force on the lateral external sidewalls of the nose in a direction which is substantially in the width direction of the nose. The arch part 30 comprises a support portion 31 adapted for supporting the adaptor means 4, when the adaptor means is interfacing with the patient's respiratory system. The arch part 30 is rotatably mounted around pivots 33 on the nose pads 33. In other words the arch part 30 is rotatably mounted along an axis defined by a straight line connecting the nose pads. The arch part 30 can freely rotate over the nose tip and can be brought to and away from a fixing position where it supports and keeps in position the adaptor means. The arch part 30 can be fixed to the adaptor means after the adaptor means has been connected to the patient's respiratory system, or it can be permanently fixed to the adaptor means. In the latter case it can be for instance rotatably mounted to the adaptor means. It can be mounted to the adaptor means near the and/or with the support portion 31. In the latter case the arch part does not necessarily need to be rotatable with respect to the nose pads. The pressure or force exerted on the lateral external sidewalls of the nose can at least partially be caused by the weight of the fastening means (comprising the weight of the nose pads 32 and the weight of the arch part 30). This force can be strengthened by providing an arch 30 which itself is or can provide a force in a direction which is substantially in the width direction of the nose. The arch part 30 can therefore for instance comprise a component of the spring type.

In FIG. 6 a force diagram is depicted illustrating the forces equilibrium for nose clamping from the outside. Nose clamping from the outside can benefit from friction on the nose against sliding off (F_{R}) and clamping or normal force (F_{N}). The result of those forces is the total force (F_{T}) which defines the pull out force for the adaptor means, ie when the force is greater then F_{T}, the clamp will loosen and the adaptor means may be released from its position. In the case for instance of continuous positive airway pressure (CPAP) patient interface devices used for instance with patients suffering from sleep apnea, the pressure will be released and the patient will awake. A firm positioning of the adaptor means is thus crucial. FIG. 6 also illustrates the placement of an adaptor means comprising two cannula into the nose, the two cannula 41 corresponding to respective nasal passages. The cannula 41 can have for instance a circular or an elliptical cross section, and are preferably at least partially, more preferably completely rigid. Preferably they reach up until the level of, or about the level of, the nose pads when interfacing the respiratory system/nose/nasal passages of the patient. Note that the preferred level is thus higher than the level depicted in FIG. 6. This way they can counteract the pressure exerted by the nose clamp, such that the nasal passages are not substantially reduced or closed off, as the cannula are hollow, i.e. they comprise a longitudinal bore through which gas can flow.

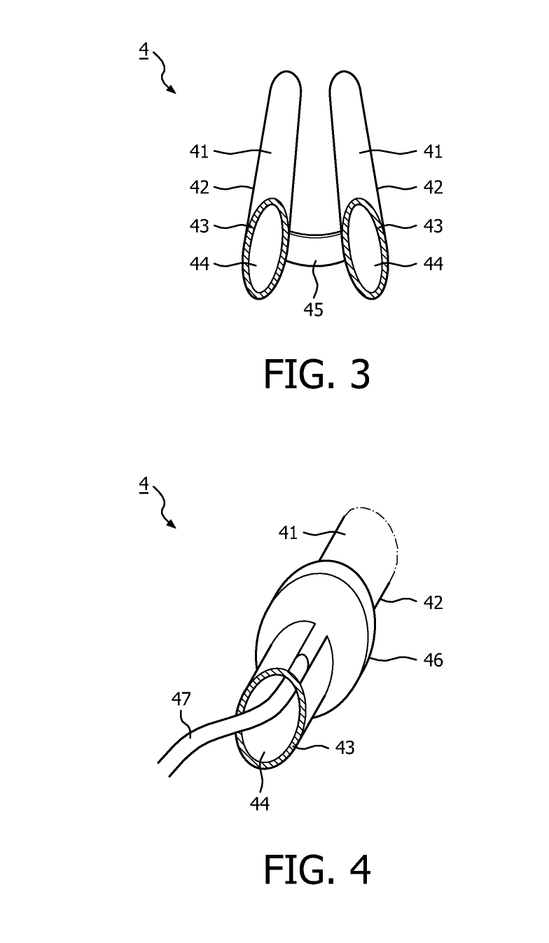

In FIG. 3 a first example is depicted of an adaptor means 4 (rigid nostril insert) according to embodiments of the present invention. The adaptor means 4 comprises two cannula 41, having a cylindrical type shape. They comprise a bore 44 defining an inner wall 43, adapted for guiding the gas flow, and have an outer wall 41 suitable for being in contact with the internal wall of the nasal passages of the patient. Therefore the outer wall is preferably smooth, i.e. free from projections or unevenness of surface and not rough. Also the internal wall is preferably smooth as it should limit easily be cleanable and should not block the airflow too much. The cannula 41 can have for instance a circular or an elliptical cross section, or can have an ergonomically adapted shape for better fitting into the nasal cavities. They are preferably at least partially rigid in order to counteract the pressure exerted by a nose clamp 3. The adaptor means (or adaptor device) can further comprise an interconnect portion 45 connecting the two cannula 41, for easier manipulation of the adaptor means. The interconnect portion is preferably located near the outer ends of the cannula 41 (the end which are pointing outwards when the adaptor means is interfacing the patient). Preferably the interconnect portion 45 can have a flat strip, i.e. a long narrow piece of material, the main surfaces of which being positioned substantially parallel to the columella of the nose. Preferably the interconnect portion comprises a surface, for instance one of the main surfaces of the strip, which is adapted for resting against the columella of the patient when the adaptor means is interfacing the patient. The flat strip can be rigid, but can also be flexible for more comfort during application.

Alternatively, the cannula 41 may, instead of being rigid, be inflatable to a certain extent. This may cause a better airtight fitting with the internal wall of the nasal passages. When inflated, the pressure should be high enough such that the pressure or force exerted by the nose clamp is sufficiently countered. Furthermore, the cannula should have a bore which is or stays large enough when inflated, in order to allow the flow of gas through them. Therefore, the cannula can for instance comprise a rigid internal wall, while the rest of the material of the cannula can be a flexible or even stretchable material.

The cannula 41, or nostril inserts, of adaptor means 4 may also comprise an inflatable cuff 46. This is illustrated in FIG. 4. The cuff 46 can improve the fitting within the nasal passages of the patient. This improvement may be more substantial when a non patient-specific or non nose-type specific insert is being used or provided to the patient.

In the above embodiments with partially or completely inflatable cannula 41, or in the case with a non inflatable cannula 41 with an inflatable cuff 46, the inflatable part may be fed by an inflation channel or tube 47. The pressure used by a positive airway pressure interface device used with the patient interfacing device, or of which the patient interface device is part, can partially serve for inflating the respective parts. Alternatively an independent inflation means may be provided.

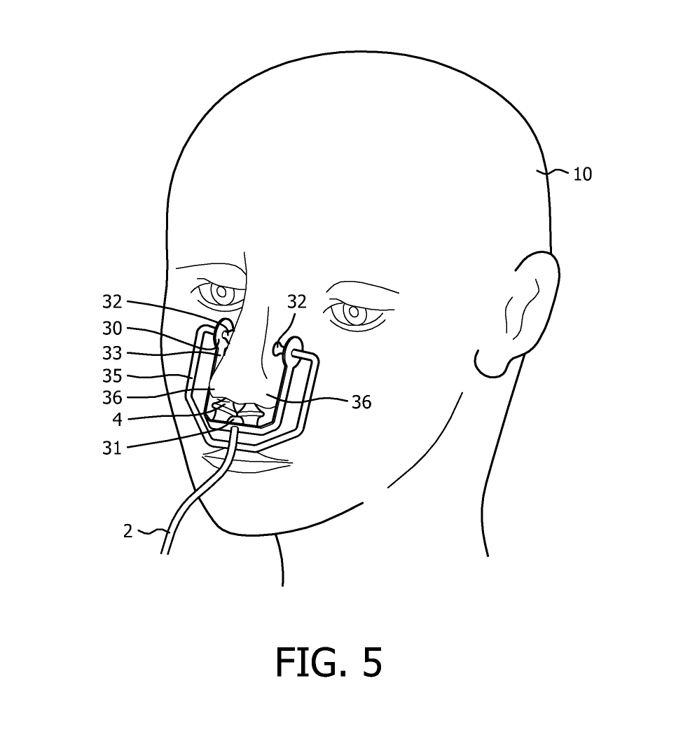

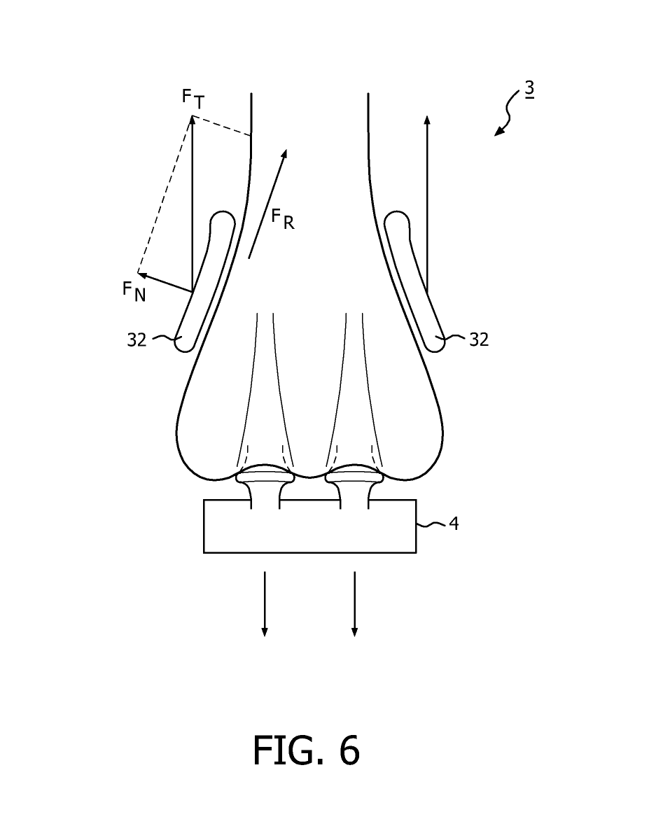

In FIG. 5 a further embodiment of the present invention is illustrated. A hose 2 is guiding a flow of gas and/or from the patient's respiratory system and coupled to an adaptor means 4. The flow of gas is guided by means of the adaptor means 4 towards the nasal passages of the patient. The adaptor means 4 can for instance correspond to the one depicted in FIG. 6. Nose pads 32 are coupled with an arch part 30 as described for FIGS. 1 and 2, wherein the arch part 30 is pivotably or rotatably mounted to the nose pads by means around pivot points 33. Here, the nose pads themselves are embodied as being inflatable. The inflation of the nose pads can result in the fixation of the fastening means 3 to the nose. Although an additional pressure or force may be exerted by a suitable arch part 30 as described above, this is not necessary as the inflation of the nose pads can cause the build-up of pressure on the nose. Air pressure can be provided to the nose pads by channels or tubes 35. As for the inflatable nostril inserts 41 or partially inflatable nostril inserts 41 with cuffs, the required pressure for inflating the nose pads can be derived from the pressure used by a positive airway pressure interface device used with the patient interfacing device, or of which the patient interface device is part. Alternatively an independent inflation means may be provided.

According to certain embodiments of the present invention some of or all of the inflatable or partially inflatable nose inserts and the inflatable nose pads can be provided with pressure by a common pressure provisioning means. The hoses 35 and 47 can thus for instance be connected. They can be connected by being in series, i.e. a single hose 35 , 47 can lead to a nose pad and to a nostril insert 41. Two different such hoses can be used for the left and right part of the patient. These hoses can also be bifurcated from a mother hose. Alternatively, a single hose can provide pressure to both nose pads 32 and both inflatable nostril inserts 41 in series or in parallel.

As described in the previous embodiments, the nose clamp or other fastening means 3 can be connected or attached to, or can interact with the external surface of the nose. In FIG. 8 a further embodiment of the present invention is depicted. The nose clamp 3 or other fastening means comprises a coupling means for coupling with a removable cap 5. The removable cap can be added once the patient interfacing device is positioned, in order to provide a more closed and smooth outer surface. The removable cap may also be adapted for guiding a combination of hoses 2 and/or 35 and or 47 or other hoses along a possibly predetermined path. The cap 5 may comprise apertures or internal guiding means for this purpose. External guiding means for hoses may also be provided to the cap, although this may introduce more irregularity on the outer surface. This may again increase comfort and manipulability of the device. The presence of such a cap surface improves the stability of the interfacing device, as it becomes more difficult penetrate or pull at any of its component with objects external to the device, as there are for instance sheet tissue or fingers of the patient. It also reduces the possibility that the patient would unconsciously remove (part) of the mask or hoses when sleeping. Advantageous the cap 5 and patient interfacing device can be coupled by a coupling mechanism which can easily be decoupled when the patient is awake, but which is difficult to decouple while the patient is asleep or unconscious.

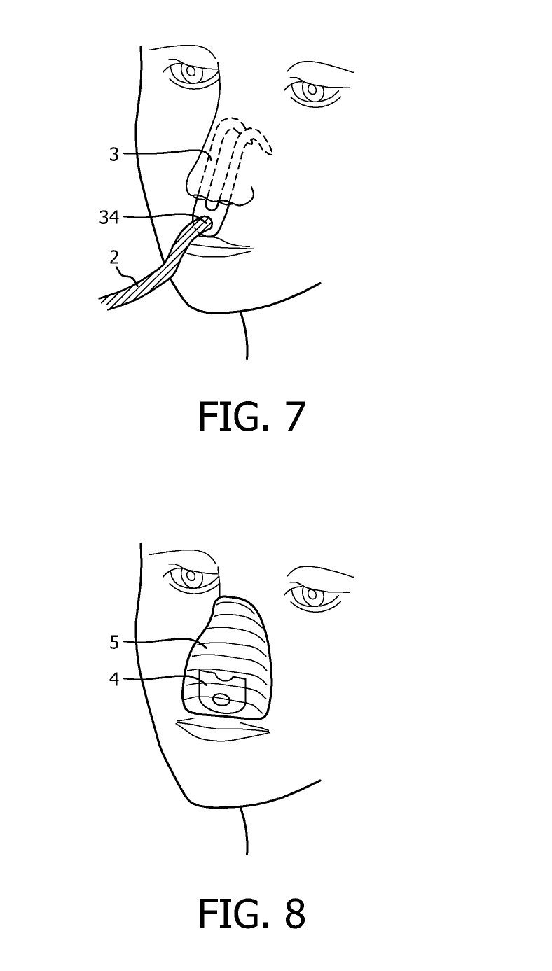

The fastening means 3 can also be attached internally in the nose. An example of such an embodiment is shown in FIG. 7. Here a fastening means 3 comprises or consists of a hook, which is adapted for being attached into the nose. The hook can for instance be adapted for hooking behind an internal feature of the nose. At the opposite end of the hook, it can be fixed to the adaptor means 4, keeping it into place. The fastening means may be suitable for being removed every time after the interfacing device has been applied to the patient. It may also remain in the patient for a longer period of time. The fastening means may further comprise an opening 34 for guiding a hose guiding the gas flow with the patient. The aperture can be positioned and adapted such that a simple coupling between a hose 2 and the adaptor means 4 can be achieved. It can for instance comprise a alignment functionality towards an ingress opening of the adaptor means 4, the latter of which can provide a guidance towards 2 egress openings, typically at the outer end of the cannula 41.

Other arrangements for accomplishing the objectives of embodiments of the present invention will be obvious for those skilled in the art.

The patient interfacing devices according to embodiments of the present invention can advantageously be used for treating patients, which suffer from sleep apnea, by using it in the context of continuous positive airway pressure (CPAP) or similar treatments.

It is to be understood that although preferred embodiments, specific constructions and configurations, as well as materials, have been discussed herein for patient interface devices according to the present invention, various changes or modifications in form and detail may be made without departing from the scope and spirit of this invention. For example, alternative types of “anchors” to the nose can be applied, as well as alternative fastening means fixed to the nose by means of these anchors. The anchors can for instance also comprise nose pads which can interact magnetically with the cannula. The fixation to the nose of the fastening means can be internal as well as external to the nose. The use of skin piercings as anchors or attachment point to the noise is not excluded. The nose pads can for instance also be ergonomically shaped in order to fit better to certain types of noses. They can for instance be made fitting perfectly to a specific nose. The contact surfaces of the nose pads for being brought in contact with the nose skin can also be adapted for providing improved friction. They can for instance be roughened or coated with an appropriate coating.

Clamping of the nose can also occur based on state of the art alternative mechanisms.

1. A patient interface device (1) for delivering a gas to a patient, the patient interface comprising a fastening means (3) for fastening the patient interface to the patient, the fastening means (3) comprises a nose clamp adapted for engaging the patient's nose during use, which nose clamp comprises nose pads (32) for being brought in contact with the lateral external sidewalls of said nose,
wherein said fastening means (3) is adapted for being fixed to said nose by exerting a pressure on said nose substantially in the width direction of said nose.

2. (canceled)

3. (canceled)

4. A patient interface device according to claim 1, wherein said nose pads (32) are inflatable, such that when they are inflated the exerted pressure on said nose is substantially larger then when they are not inflated.

5. A patient interface device (1) according to claim 1, further comprising an adaptor for providing a coupling between a gas supply (2) and the nasal passages of said respiratory system, and wherein said fastening means (3) is adapted for fixing the position of said adaptor (2).

6. A patient interface device according to claim 5, further comprising an arch part (30) which is on its outer ends connected to said nose pads (32), and wherein said arch part (30) further comprises a support portion (31) adapted for supporting said adaptor (4).

7. A patient interface device according to claim 6, wherein said arch part (30) is rotatably mounted along an axis defined by a straight line connecting said nose pads (32), such that it can freely rotate over a tip of said nose, and such that it can be brought in a fixing position below said nose where it can support said adaptor (4).

8. A patient interface device according to claim 6, wherein said arch part (30) is fixed to and rotatably mounted to said adaptor (4) near its support portion.

9. A patient interface device according to claim 6, wherein said arch part (30) is mainly rigid or semi rigid, except for its outer ends which are abutting on said nose pads or forming said nose pads, said outer ends being flexible and elastic.

10. A patient interface device according to claim 6, wherein said adaptor (4) comprises a pair of cannula (41) to be inserted into the nasal passages of the patient, said cannula (41) being at least partially rigid, said nose clamp (3) and said cannula (41) being adapted for cooperating in positioning and fixing said adaptor (4).

11. A patient interface device according to claim 10, wherein said cannula (41) are connected by an interconnect portion (45), an upper surface of which is adapted for resting against the columella of the nose.

12. A patient interface device according to claim 10, wherein said cannula (41) are at least partially inflatable.

13. A patient interface device according to claim 10, further comprising a removable cap (5) covering said fastening means (3).

14. A patient interface device according to claim 1, wherein said fastening means (3) comprises a structure (30) for being attached within said nose.

15. (canceled)

16. A patient interface device according to claim 5, wherein each cannula (41) comprises an inflatable cuff.

17. A patient interface device according to claim 10, wherein each cannula (41) comprises an inflatable cuff.

18. A patient interface device according to claim 17, wherein each inflatable cuff comprises a cuff aperture in the cannula (41) such that the cuff can be inflated by a positive airway pressure generated during inhalation and exhalation.
